(19) United States
(12) Patent Application Publication
(10) Pub. No.: US 2007/0043440 A1
(43) Pub. Date: Feb. 22, 2007

(54) Method and apparatus for treating diseased or fractured bone

(76) Inventors: Michael S. William, Santa Rosa, CA (US); Jeffrey A. Smith, Santa Rosa, CA (US); Richard S. Stack, Chapel Hill, NC (US); Kevin D. Holbrook, Cloverdale, CA (US); Richard A. Glenn, Santa Rosa, CA (US)

Correspondence Address: DEANNA J. SHIRLEY, 3418 BALDWIN WAY, SANTA ROSA, CA, 95403, US

(21) Appl. No.: 10/571,580

(22) PCT Filed: Sep. 17, 2004
(86) PCT No.: PCT/US04/30771

§ 371 Date: Mar. 10, 2006

Related U.S. Application Data
(60) Provisional application No. 60/504,333, filed on Sep. 19, 2003.

(51) Int. Cl. A61F 2/44

(52) U.S. Cl. 623/17.11

(57) ABSTRACT

An apparatus according to the invention may comprise one or more expandable structural support pylons (140), means for deploying said one or more structural support pylons (138, 141), and one or more therapeutic substances. Structural support pylons (20) may comprise an elastomeric core (22), or may comprise one or more suitable metals, and may be deployed by shortening the length and increasing the height of the pylon. Alternatively, scaffold (200) may comprise a delivery configuration in which first unit (202) and second unit (204) he generally end to end, and a deployed configuration in which superior surfaces (216) and (218) exert an upward force and inferior surfaces (220) and (222) exert a downward force on superior and inferior vertebral surfaces respectively. A method according to the invention may comprise accessing an interior of a diseased or injured bone in a minimally invasive manner, creating a semi-circular path within said bone, deploying one or more structural support pylons within said interior, introducing structural reinforcement material, and if desired, repeating with one or more subsequent structural support pylons.

### FIELD OF THE INVENTION

The invention herein relates generally to medical devices and methods of treatment, and more particularly to devices and methods used in the restoration and/or repair of diseased or injured bone.

### BACKGROUND OF THE INVENTION

Osteoporosis, literally “porous” bone, is a disease characterized by low bone mass and density, and structural deterioration of bone tissue. Osteoporosis leads to bone fragility; increased susceptibility to fractures including compression fractures; neural compression; insufficient vertical support by the spine; and pain. According to the National Osteoporosis Foundation, osteoporosis is a major public health threat for an estimated 44 million Americans. According to the International Osteoporosis Foundation, osteoporosis is responsible for more than 1.5 million fractures annually, including approximately 700,000 vertebral fractures, as well as numerous fractures of the hip, wrist, and other sites.

Vertebral fractures are the most common osteoporotic fracture. Approximately 20-25% of women over the age of 50 have one or more vertebral fractures. Once a woman suffers a first vertebral fracture, the shift in force transmission upon all vertebrae result in a five-fold increase in the risk of developing a new fracture within one year. Vertebral fractures, like hip fractures, are associated with a substantial increase in mortality among otherwise relatively healthy older women. Following such fractures, treatment that requires attachment of pins, screws, or similar devices to the vertebral bodies may not be feasible because of the underlying instability of the diseased bone. Osteoporosis and vertebral fractures are further characterized by decreased height, and often collapse, of the vertebral bodies. Such decrease leads to stooped posture, decreased lung capacity, impaired mobility, neural compression, and pain.

Other disease processes, including tumor growth, especially round cell tumors, avascular necrosis, and defects arising from endocrine conditions also result in a weakened condition and/or fractures. Such other conditions, whether in the vertebrae or at other sites, are also causes of significant pain and reduced mobility in patients.

Methods for reinforcing diseased and/or fractured bone, and attempts to restore vertebral height, are known in the art. Such methods include procedures in which a health care provider may direct a filling material into the bone. Such a material, initially in a flowable state, fills fissures and/or openings within the diseased or injured bone and cures to form a hardened material that provides support to the bone. Limitations of such a procedure include overflow of material into the spinal column and inadequate support of the bone. Currently, such filling or fusion material is not approved in the United States for injection into the vertebral body. Therefore, an alternative and more reliable procedure is needed.

It is therefore an object of the present invention to provide a method of stabilization and repair of diseased and/or injured bone, whether osteoporotic or not. It is a further object of the invention to restore vertebral bodies to a normal height. It is a further object of the invention to achieve elimination of translational compression of adjacent vertebrae, decompression of nerve tissue, and reduction of pain. And finally, it is an object of the invention to achieve improved patient posture and mobility.

### SUMMARY OF THE INVENTION

An endoprosthesis for use in the treatment of diseased or injured bone comprising an elastomeric core, a reduced diameter configuration and an expanded diameter configuration, capable of withstanding multidirectional compressive loads as high as 6000 Newtons. The endoprosthesis may comprise therapeutic substances that may be disposed about the endoprosthesis via a solvent in a supercritical state. The endoprosthesis comprises a generally ellipsoidal configuration when in its deployed state, and is harder following deployment than prior to deployment. The post-deployment hardness is in the range of between 20 and 70 Shore A durometer.

The elastomeric core may comprise an aperture therethrough that may be disposed centrally or eccentrically. The endoprosthesis may be generally ellipsoidal, or may have flat sides, or may be ovular. The aperture may comprise a smooth, threaded, notched or ratcheted interior for engagement with a corresponding member. The endoprosthesis may comprise a hollow interior and/or endoprosthesis members and endplates, or may be of a braided and/or a locking braid configuration. The endoprosthesis may be used singly or in plurality, and may be disposed in an ofset manner with respect to one another. The plurality may be disposed in one group or in more than one separate groups.

The endoprosthesis may comprise a plurality of folded discs and deploy to comprise a plurality of stacked discs. The endoprosthesis may comprise a substantially elongated device that deploys to comprise a device with superior and inferior surfaces at right angles to the sides, and may expand to one and one tenth to ten times its delivery configuration height The endoprosthesis may be part of an assembly that comprises an actuating arm and structural reinforcement material.

A method for repairing diseased or injured bone may comprise percutaneously introducing an endoprosthesis comprising a generally cylindrical or elongated delivery configuration and a generally ellipsoidal deployed configuration. The method may comprise creating a generally semicircular path prior to introducing the endoprosthesis. The generally semicircular path may be either generally parallel to or generally perpendicular to the vertical axis of the spine. The method may comprise the step of introducing structural reinforcement material. The method may comprise shortening the distance between the ends of the endoprosthesis thereby increasing the height of the endoprosthesis.

### BRIEF DESCRIPTION OF THE DRAWINGS

FIG. 1 is a perspective view of healthy human vertebrae.

FIG. 2 represents a perspective view of a portion of a human spine in which a vertebral body has collapsed.

FIG. 3 is a side view of an embodiment according to the invention, shown in its delivery configuration.

FIG. 4 is a cross-sectional side view of the embodiment of FIG. 3.

FIG. 5 is a side view of the embodiment of FIG. 3 following deployment

FIGS. 6A-6B illustrate a side view of an alternative embodiment according to the invention in its delivery configuration and its deployed configuration.

FIGS. 7 is a cross sectional side view of an alternative embodiment according to the invention.

FIG. 8A is a side view of yet another embodiment according to the invention, shown in its delivery configuration.

FIG. 8B is a cross section of a member of the embodiment of FIG. 8A.

FIG. 8C illustrates the embodiment of FIG. 8A in its deployed configuration.

FIG. 8D illustrates the structure of yet another alternative embodiment of a device suitable for use according to the invention in its deployed configuration.

FIGS. 9A-9B illustrate the structure of and a sequence of steps in the deployment of another alternative embodiment of a device suitable for use according to the invention.

FIGS. 10A-10B illustrate a side view of a device according to the invention following sequential steps in deployment of the device.

FIGS. 11A-11C illustrate a side view of a vertebral body following sequential steps of treatment according to the invention, and sequential steps of deployment of a device according to the invention.

FIG. 12 illustrates an alternative embodiment according to the invention

FIGS. 13A-13B illustrate sequential steps of an alternative method and deployment of an alternative embodiment according to the invention.

FIGS. 13C-13D are additional depictions of the embodiment illustrated FIGS. 13A-13B.

FIGS. 13E-13E are also additional depictions of an embodiment similar to that illustrated in FIGS. 13A-13B.

FIG. 14A illustrates a side view of an alternative embodiment according to the invention in its deployed configuration,

FIG. 14B-14C illustrate end views of the elements used in the manufacture of the embodiment of FIG. 14A

FIG. 15A illustrates a side view of an alternative embodiment according to the invention in its deployed configuration.

FIGS. 15B-15C illustrate end views of the elements used in the manufacture of the embodiment of FIG. 15A

FIG. 16A illustrates a side view of an alternative embodiment according to the invention in its deployed configuration.

FIGS. 16B-16C illustrate end views of the elements used in the manufacture of the embodiment of FIG. 16A

FIG. 17 illustrates yet another alternative embodiment according to the invention.

FIGS. 18A-18E illustrate a side view of yet another alternative embodiment according to the invention following sequential steps in deployment

FIGS. 19A-19B illustrate perspective views of yet another alternative embodiment according to the invention in its delivery configuration and in its deployed configuration.

FIGS. 20A-20B illustrate yet another embodiment according to the invention mounted upon a mandrel, in both its delivery configuration and its deployed configuration.

FIG. 20C illustrates an alternative embodiment according to the invention in its deployed configuration

### DETAILED DESCRIPTION OF THE INVENTION

“Vertebroplasty” is a procedure used to augment diseased and/or fractured vertebral bodies, in which a biocompatible cement or filling material is infused into the vertebral body through a large bore needle under fluoroscopic guidance.

“Kyphoplasty” is a procedure similar to vertebroplasty, with the added step of creating space within the vertebral body and restoring vertebral height with the use of a balloon prior to injecting biocompatible cement or filling material.

“Spinal unit” refers to a set of the vital functional parts of the spine including a vertebral body, endplates, facets, and intervertebral disc.

The phrase “decompressing the bone” refers to a process during treatment according to the invention by which a collapsed portion of diseased or injured bone is at least temporarily restored to a near normal geometry in order that said near normal geometry may be more permanently restored.

In some embodiments according to the invention, devices referred to as “structural support pylons” are utilized. Structural support pylons used according to the invention herein may be of any suitable design, and may be fabricated from one or more conventional or shape memory alloys, polymers, or other suitable materials selected for molecular weight, chemical composition and other properties, manufactured to achieve any desired geometries and processed to achieve sterilization, desired geometries and in vivo lifetime. Structural support pylons used according to the invention may also comprise a substantially cylindrical structure, whether substantially solid or, hollow, or may be substantially ellipsoidal, spherical, or may comprise support surfaces at opposing ends of an extendable connecting member, and may comprise endplates at opposing ends of the structure. Structural support pylons used according to the invention may also comprise a generally cylindrical structure in a delivery configuration and may comprise a more ellipsoidal structure when in a deployed configuration.

A “structural reinforcement material” used according to the invention may be substantially solid, or may initially be flowable and then cure over time, or may be cured according to any number of means known in the art, including, but not limited to, by chemical reaction or following exposure to an energy source. Suitable structural reinforcement materials include, but are not limited to expandable polyurethane foam, poly-methyl-methacrylate (PMMA), catalytically reactive PMMA, calcium carbonate, calcium phosphate, oxalate, polyglycolic acid, polylactic acid compounds, shape memory polymers including but not limited to polyurethane, polyethylene, high density polyacrylamide, cyanoacrylates, hydroxyapatite derivatives, collagen, chitin, chitosan, silicon, zirconium, and others suitable for providing structural reinforcement and/or stabilizing the vertebral body. Commercial preparations such as Osteobond, available from Zimmer, Inc., of Warsaw, Indiana, or Howmedica Simplex from Stryker Corporation of Kalamazoo, Michigan, are also suitable. Radiopacity can be enhanced in any of the foregoing with the addition of particles comprising barium, barium sulfate, bismuth trioxide, tantalum, tungsten, zirconium, gold, platinum, platinum iridium, stainless steel, or other radiopaque material.

An “expandable” endoprosthesis comprises a reduced profile configuration and an expanded profile configuration. An expandable endoprosthesis according to the invention may undergo a transition from a reduced configuration to an expanded profile configuration via any suitable means, or may be self-expanding.

The term “fiber” refers to any generally elongate member fabricated from any suitable material, whether polymeric, metal or metal alloy, natural or synthetic.

The phrase “points of intersection”, when used in relation to fiber(s), refers to any point at which a portion of a fiber or two or more fibers cross, overlap, wrap, pass tangentially, pass through one another, or come near to or in actual contact with one another.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

As used herein, the term “braid” refers to any braid or mesh or similar woven structure produced from between 1 and several hundred longitudinal and/or transverse elongate elements woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric.

A “ratchet column” is a toothed bar the teeth of which slope in one direction so as to catch and hold a pawl or other engaging unit, thus preventing movement in a reverse direction.

Though not limited thereto, some embodiments according to the invention comprise one or more therapeutic substances that will elute from the surface. Suitable therapeutics include but are not limited to bone morphogenic protein, growth factors, osteoconductive agents, and others. According to the invention, such surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”).

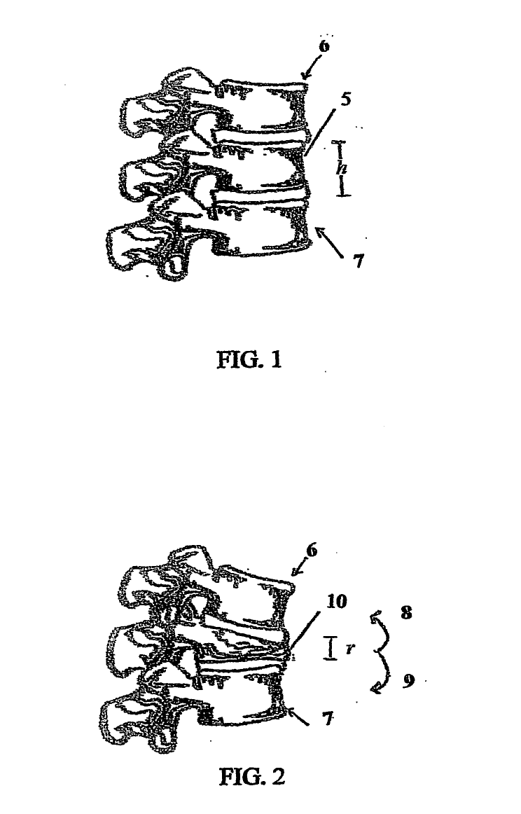

Details of the invention can be better understood from the following descriptions of specific embodiments according to the invention In order to illustrate, a spine with healthy vertebrae is represented in FIG. 1. Healthy vertebral body 5 displays normal vertebral height h. The axial load normally incident on a healthy spine is distributed in a relatively balanced fashion among the vertebrae pictured.

In FIG. 2, a spine having a fractured vertebral body 10 is illustrated. Fractured vertebral body 10 is shown to have reduced vertebral height r. In addition to reduced vertebral height r, the previous relatively even distribution of axial load among the vertebrae pictured has been disrupted. Load previously borne in a balanced fashion by fractured vertebral body 10 is now transmitted to vertebral bodies 6 and 7, in the direction of arrows 8 and 9. Further, the orientation of vertebral bodies 6, 10 and 7 with respect to one another has shifted in correspondence with the change in geometry of vertebral body 10. The increase and shift in loads on vertebral bodies 8 and 9 increases the likelihood of fracture and/or collapse of vertebral bodies 8 and 9, especially in a patient suffering osteoporosis. Methods for repairing or restoring vertebral body 10 and devices used according to such methods are described in detail below.

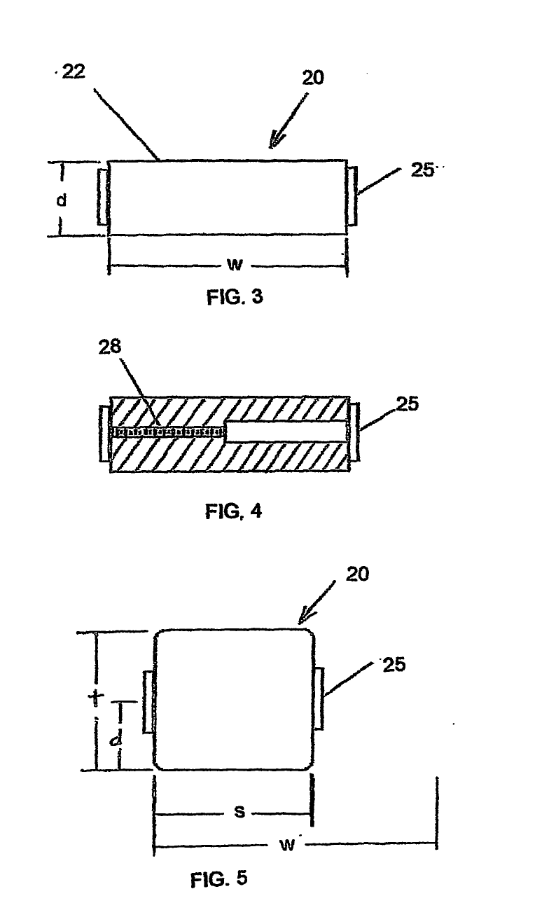

Embodiments according to the invention and suitable for use according to one or more methods of the invention are illustrated in FIGS. 3-9B and 11A-16. FIGS. 3-5 illustrate an embodiment according to the invention Structural support pylon 20, shown from a side view in FIG. 3 and in cross section in FIG. 4, comprises elastomeric core 22, disposed between endplates 25. Endplates 25 further comprise threaded element 28 extending therebetween, as revealed in FIG. 4. In FIGS. 3 and 4, structural support pylon 20 is in its delivery configuration, which is generally cylindrical, having height d and width w. The delivery configuration facilitates introduction of structural support pylon 20 to a treatment site in a minimally invasive manner. Following delivery to a treatment site, structural support pylon 20 is placed in its deployed configuration by advancing endplates 25 over threaded element 28, drawing endplates 25 closer to one another, and compressing elastomeric core 22 along its width w, thereby increasing height d of elastomeric core 22.

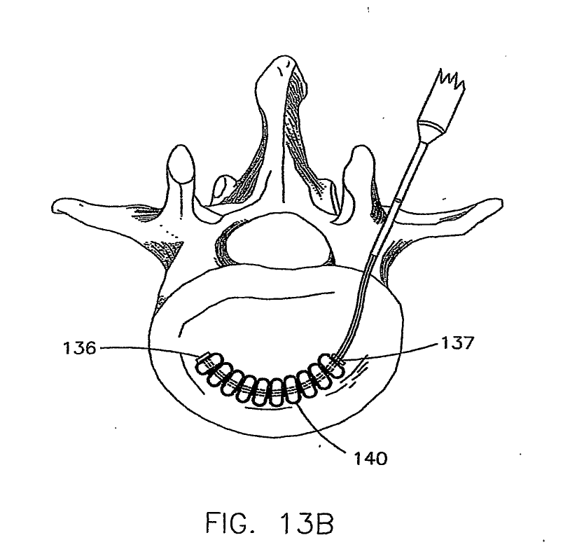

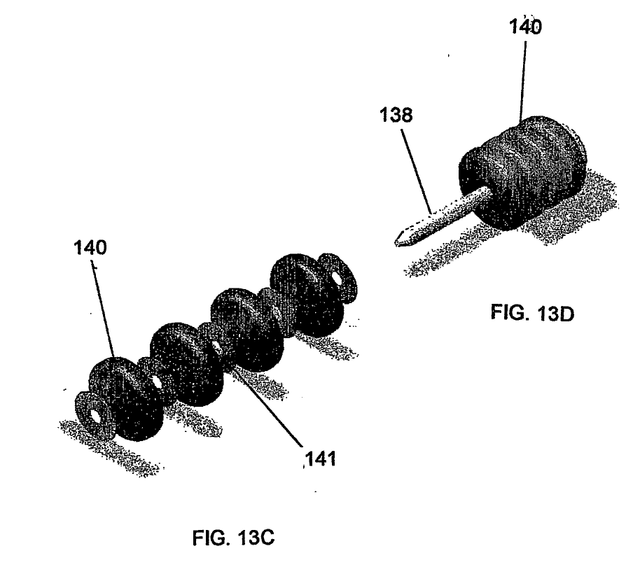

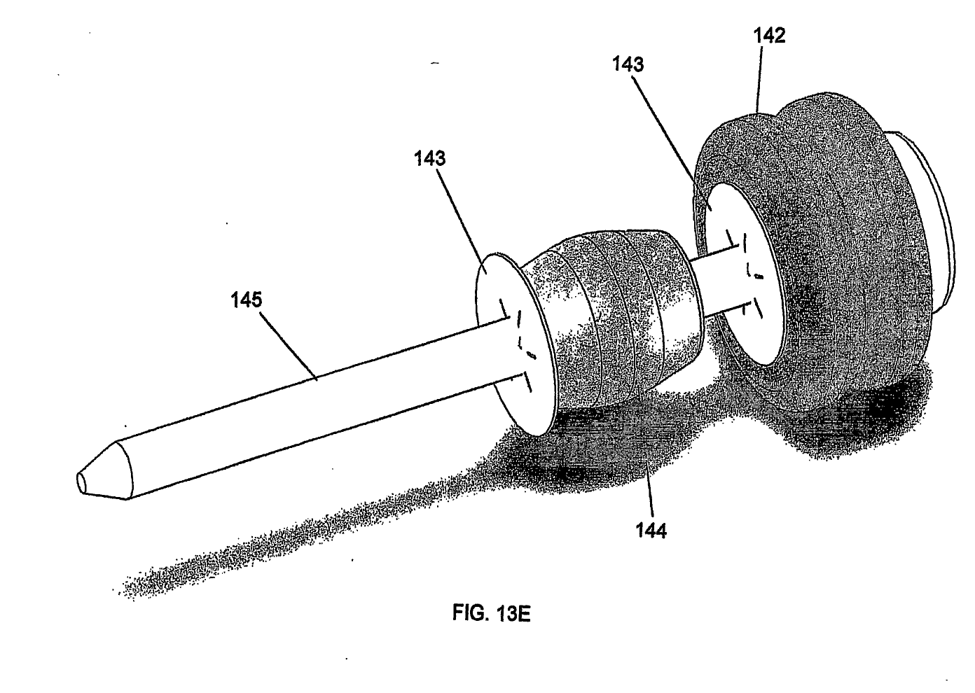

As shown in FIG. 5, structural support pylon 20, in its deployed configuration, comprises increased height t, and decreased deployed (or implanted) width s. Increased height t is typically roughly between one and one tenth (1.1) times and eight (8) times height d. Structural support pylon 20 may be generally spherical or generally ellipsoidal in its deployed configuration. Further, the hardness of structural support pylon 20 or 30 increases from an initial hardness of as little as approximately 10 Shore A durometer to a post deployment hardness of 70 Shore A, due to the compressive axial load. The increased hardness, coupled with the radial expansion described above, exerts a radial force and provides a body capable of withstanding columnar forces exerted on the vertebral body. The embodiment of FIGS. 3-5 or similar embodiments may be utilized singly, or alternatively, two or more may be utilized together, as discussed in greater detail in relation to FIGS. 13A-16C.

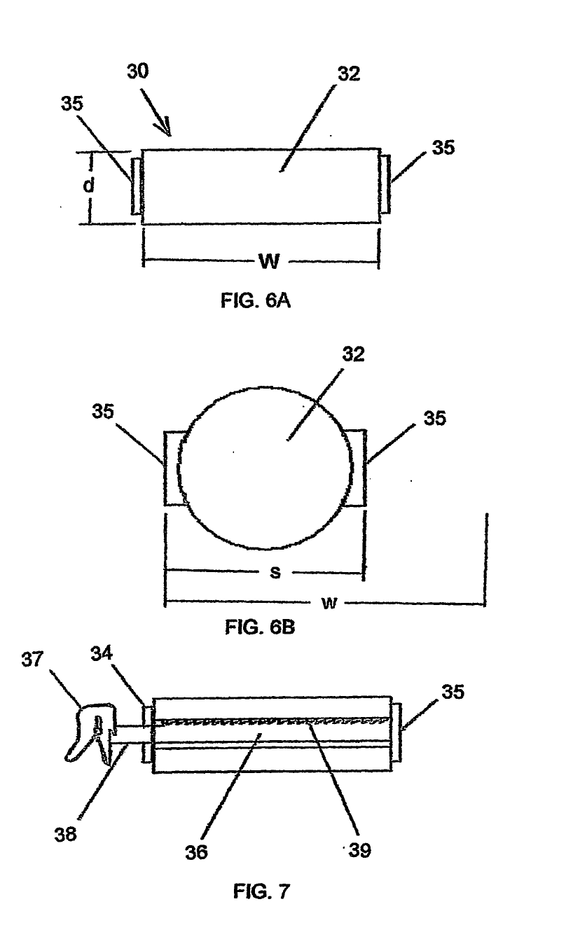

FIGS. 6A-6B illustrate an embodiment similar to that of FIGS. 3-5. Structural support pylon 30 comprises elastomeric core 32, endplates 35, delivery configuration height d, and delivery configuration width w. By drawing endplates 35 closer to one another, the configuration of structural support pylon 30 transitions from its delivery configuration, shown in FIG. 6A, to its deployed configuration, shown in FIG. 6B. In its deployed configuration, structural support pylon 30 comprises increased deployed configuration height t and decreased deployed configuration width s. Structural support pylon 30 may comprise either a generally more ellipsoidal shape or a generally spherical shape when in its deployed configuration.

In order to maintain its deployed configuration, structural support pylon 30 comprises ratchet column 36 extending between endplates 35, as shown in cross section in FIG. 7. Ratchet column 36 comprises ratchet handle 37, ratchet bar 38 and a plurality of teeth 39. Upon advancement of ratchet bar 37 via ratchet handle 37, endplates 35 are irreversibly drawn closer to one another as teeth 39 engage with ratchet column engagement member 34.

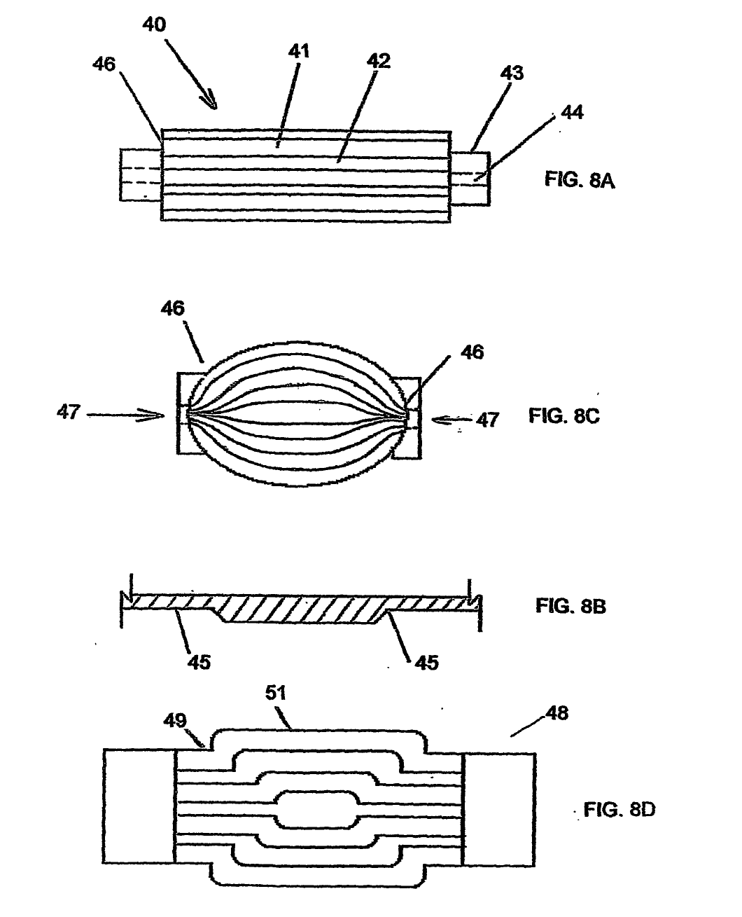

FIGS. 8A-8C illustrate yet another embodiment of a device suitable for use according to the invention. FIG. 8A is a side view of structural support pylon 40 in its generally cylindrical delivery configuration comprising pylon members 41, slots 42, end portions 43, and threaded holes 44. As shown in FIG. 8B, pylon members 41 comprise tapered regions 45 proximate end portions 43. Tapered regions 45 consequently constitute preferential bending regions 46. In order to deploy structural support pylon 40, an actuating tool (not pictured) is introduced via and engages threaded holes 44, and end portions 44 are drawn closer to one another in the direction of arrows 47. Meanwhile, pylon members 41 bend at preferential bending regions 46, and structural support pylon 40 transforms from a generally cylindrical configuration into a generally ellipsoidal shape between end portions 44.

The embodiment depicted in FIG. 8D is similar to that illustrated in FIG. 8C. Structural support pylon 48 comprises preferential bending regions 49 of pylon members 51, facilitating the transition to the deployed configuration shown.

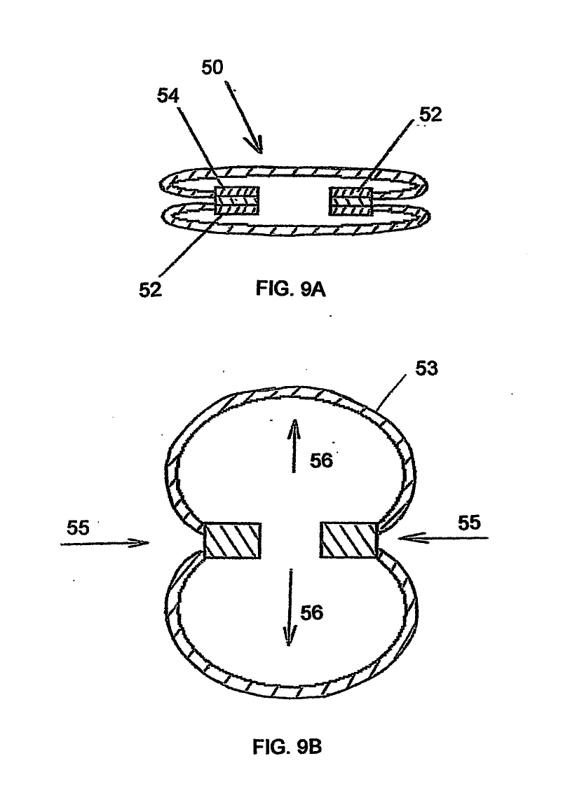

Alternatively, an inverted sleeve device such as that set forth in FIGS. 9A-9B may be employed according to the invention. Structural support sleeve 50, shown in its generally cylindrical delivery configuration in FIG. 9A, comprises inverted end members 52. End members 52 comprise threaded arm 54, shown in cross section in FIG. 9A and engage sleeve 50 within its interior. An actuating tool (not pictured) may be utilized to advance threaded arm 54, thereby tightening ring 53 in the direction of arrows 55. Tightening of sleeve 53 in the direction of arrows 55 in turn causes sleeve 53 to expand in the direction of arrows 56. Similar to the foregoing embodiments, structural support pylon 52 correspondingly undergoes a transition from its delivery configuration to its deployed configuration. The foregoing embodiments typically undergo between a one and one tenth (1.1) fold and an eight (8) fold increase in height during deployment.

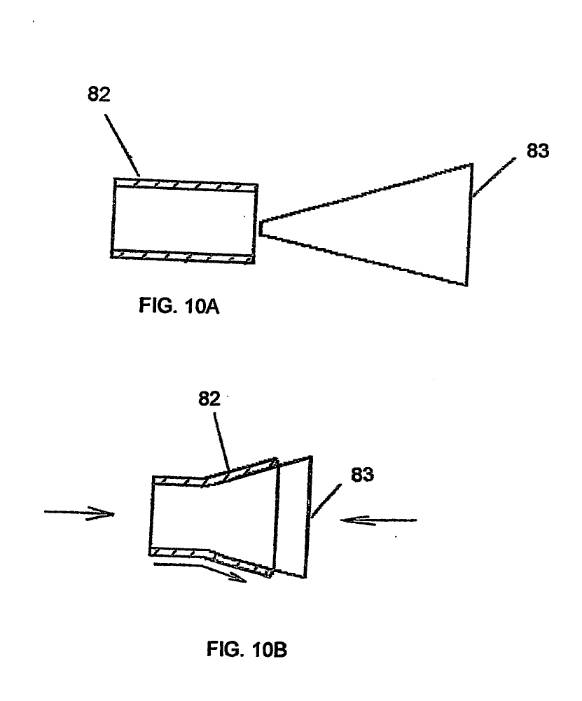

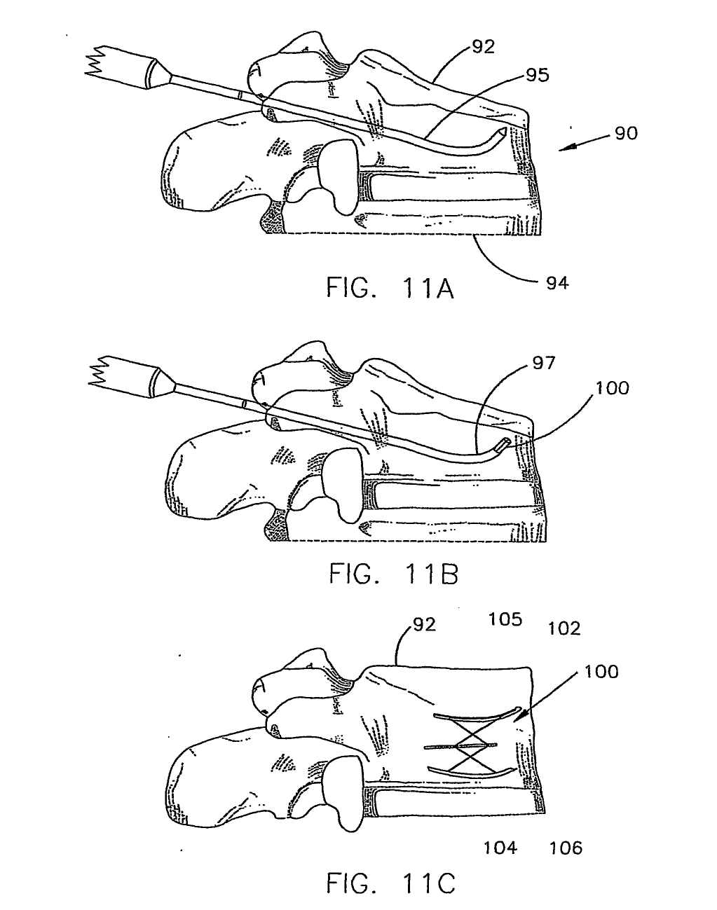

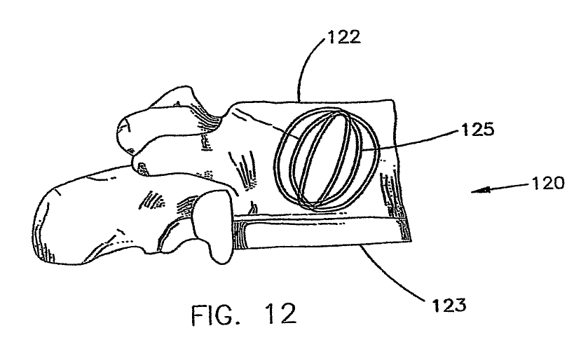

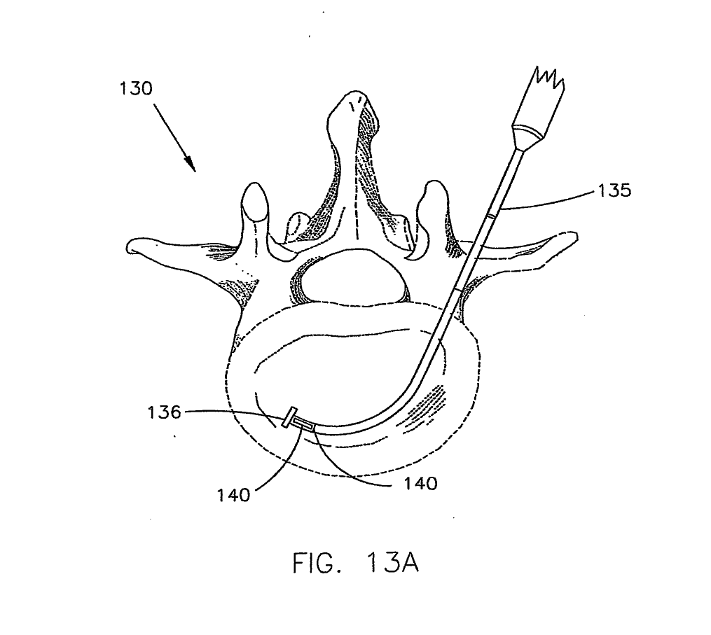

Alternatively, a generally cylindrical structural support pylon comprising open ends may be used in a method according to the invention. Accordingly, a generally cylindrical structural support pylon may be expanded by mechanical means, for example, as depicted in FIGS. 10A-10B. As illustrated in the example set forth in FIGS. 10A-10B, structural support pylon 82, shown in cross section, may be expanded in a step-wise fashion by drawing tapered plug 83 into structural support pylon 82, or, stated otherwise, by drawing structural support pylon 82 over tapered plug 83. Such a device may be used singly or in conjunction with two or more devices following a method according to the invention

Turning now to FIGS. 11A-11C, a method of treatment and an alternative embodiment according to the invention are disclosed. FIG. 11A represents a side view of collapsed vertebral body 90. Within collapsed vertebral body 90, flexible, steerable trocar 95 has been introduced in a minimally invasive manner. Flexible, steerable trocar 95 may be mounted on a flexible cable that is steerable in 360 degrees, and introduced in a posterior orientation, initially in the direction of inferior vertebral body endplate 94, and then turning toward superior vertebral body endplate 92.

Flexible, steerable trocar 95 thereby creates a channel in a generally curvilinear path defining a semicircle roughly parallel to the vertical plane as viewed from the side as in FIGS. 11A-11C. (An alternative path according to an alternative embodiment of the invention is illustrated in FIGS. 13A-13B below.) A flexible drill or curved obturator or awl (not pictured) may be introduced percutaneously in order to displace the fractured bone material within collapsed vertebral body 90 along a path established by flexible steerable trocar 95.

As illustrated in FIG. 11B, following the creation of a path as set forth above, flexible cannula 97, bearing vertebral body jack 100 (in its delivery configuration), may then be introduced within collapsed vertebral body 90. Once flexible cannula 97 has been placed within a desired position, a pushing mandrel (not pictured) within cannula 97 operates to force vertebral bodyjack 100 out of the distal end of flexible cannula 97. Once vertebral body jack 100 is displaced from the interior of flexible cannula 97, vertebral body jack 100 transitions to its deployed configuration, as shown in FIG. 11C. As vertebral bodyjack 100 converts to its deployed configuration, superior prosthesis end plate 105 exerts a force against superior vertebral body end plate 92. Inferior prosthesis end plate 106 simultaneously exerts an outward force, bracing against inferior vertebral body end plate 94, thereby providing significant vertical force, and substantially stabilizing vertebral body 90 and/or restoring vertebral body to a normal height, as shown in FIG. 11C.

Jack support members 102 comprise threaded connecting member 104, which may be actuated with a driving mechanism within cannula 97. Alternatively, jack support members 102 may be manufactured from conventional shape memory materials programmed to revert to a deployed configuration upon delivery from the distal end of cannula 97, and to exhibit extensive vertical support. As a further alternative, jack support members 102 may comprise a ratcheting connecting member that may be actuated with a tool within a cannula to deploy jack support members 102.

Turning now to FIG. 12, an alternative embodiment according to the invention is illustrated in its deployed configuration within a vertebral body. Following either the delivery path described above in relation to FIGS. 11A-11C, or, in the alternative, the delivery path described below in relation to FIGS. 13A-13B, basket 125 is delivered and deployed. Basket 125 comprises concentric rings 127 and a generally spherical, ellipsoidal, toroidal, or other suitable configuration. Basket 125 may be covered with a biocompatible elastomeric membrane and/or filled with a biocompatible polymer or other suitable structural support material. Basket 125 exerts a multi-dimensional, and especially vertical outward force against superior vertebral body end plate 122 and inferior vertebral body end plate 123. The embodiments depicted in FIGS. 11C and 12 most often undergo an increase in height of between one and one tenth (1.1) times and ten (10) times their delivery configuration height.

As noted above, the embodiments of FIGS. 3-5 can be used alone or in multiples. FIG. 13A illustrates an alternative method according to the invention, in which a plurality of structural support members 140 may be utilized. FIG. 13A is atop view of vertebral body 130. According to the method illustrated in FIGS. 13A-13B, a flexible, steerable trocar (not pictured) is introduced posteriorly in a minimally invasive fashion and directed substantially laterally through the interior of vertebral body 130, generally following the curve of the anterior portion of vertebral body 130, perpendicular to the substantially vertical axis of the spinal column. Following the percutaneous creation of a path through the interior of vertebral body 130 using a flexible steerable trocar, an obturator or cutting tool (not pictured) may optionally be used to displace diseased and/or injured bone.

As shown in FIG. 13A, flexible steerable cannula 135 for delivery of a plurality of structural support pylons 140 is introduced along a suitable path created as set forth above. Once flexible steerable cannula 135 has been placed in a desired position within vertebral body 130, structural support pylon 140 is advance to mandrel stop 136. A pusher tube (not pictured) forces and affixes washer 141 against structural support pylon 140. The force exerted upon structural support pylons 140 reduces the length of pylon 140 and increases its circumference and hardness. The hardness increases from an initial hardness of as little as approximately 10 Shore A durometer to a post deployment hardness of 70 Shore A, due to the compressive axial load. Structural support pylon 140 expands radially, exerting a radial force and providing a body capable of withstanding columnar forces exerted on the vertebral body.

Further, structural support pylon 140 transitions from a generally cylindrical configuration to a generally ellipsoidal or, alternatively, a generally round configuration. (Such a transition is described above with respect to an individual structural support pylon in FIGS. 3-8.) The distinct configurations and ease of deployment allows it to be delivered in a non-invasive fashion and then to provide substantial support within the interior of a diseased and/or injured vertebral body. The foregoing procedure is repeated for each subsequent washer 141 and structural support pylon 140. The plurality of deployed structural support pylons 140, with an unseen washer 141 between each, and final washer 137 are shown in FIG. 13B.

For clarification, FIG. 13C is an exploded view of alternating structural support pylons 140 and washers 141. When actually deployed on mandrel 138, as shown in FIG. 13D, washers 141 cannot be seen between each structural support pylon 140. Similarly, FIG. 13E clarifies the predeployed and deployed configurations of structural support pylons and the position of alternating washers. Structural support pylons 142 have been compressed to undergo deployment, and alternate with washers 143. Structural support pylon 144 has not yet been deployed. Structural support pylon 144 will be further advanced over mandrel 145 to abut each washer 143. Proximal washer 143 will then be advanced over mandrel 145, shortening the distance between each washer 143 and compressing structural support pylon 144 until it achieves the configuration of deployed structural support pylons 142.

Structural support pylons 140 may be manufactured from a suitable polymeric material. Optionally, structural support pylons 40 may comprise polymeric materials that exhibit strain induced crystallization, thereby further increasing the hardness of the material upon compression. Suitable material may also be composites of polymers, silicones, rubbers, metals and other natural and synthetic materials.

An alternative method to that illustrated in FIGS. 13A and 13B, one or more structural support pylons may be introduced into the interior of a vertebral body from two separate access points. One or more structural support pylons can be positioned contralateral to one another, from a first access point as illustrated in FIG. 13A, and from the corresponding access point at the opposite side of a vertebral body in a subsequent step.

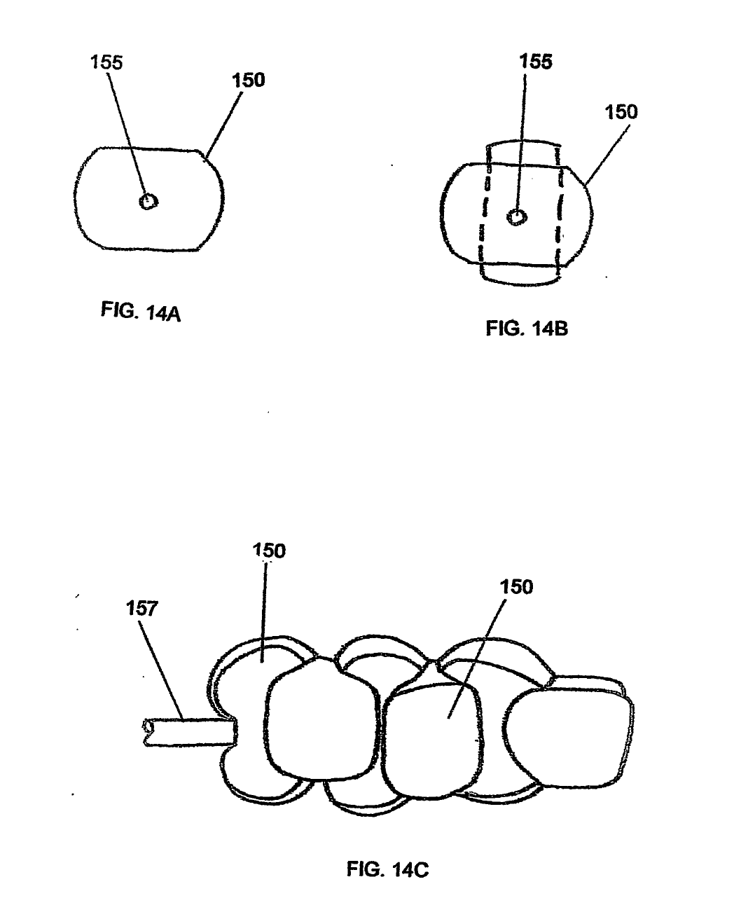

According to the invention, the plurality of structural support pylons may comprise alternative configurations. For example, structural support pylon 150, shown singly in an end view in FIG. 14A, comprises a generally central aperture 155. When mounted on mandrel 157, structural support pylons 150 may be offset with respect to one another at between 45-degree and 90-degree angles, as shown in an end view in FIG. 14B. FIG. 14A illustrates the appearance of such a configuration following deployment. Because each structural support pylon 150 is offset with respect to each adjacent structural support pylon, each can achieve a greater expansion in a given direction, resulting in a larger overall diameter device. As in the embodiments set forth above, the hardness of structural support pylon increases upon deployment.

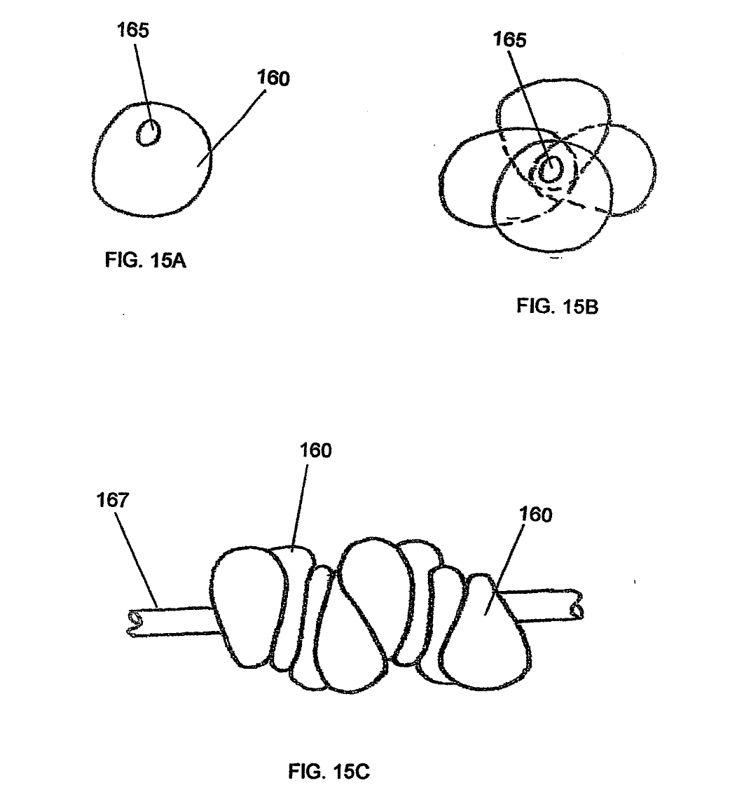

An alternative configuration is illustrated in a similar fashion in FIGS. 15A-15C. Structural support pylon 160, comprising eccentric aperture 165, is shown singly in an end view in FIG. 15A. A plurality of structural support pylons 160 may be mounted upon mandrel 167, offset with respect to one another at between 45-degree and 90-degree angles, as shown in an end view in FIG. 15B. Given the offset of each individual structural support pylon 160 with respect to one another, each can be expanded to a greater extent in a given direction, resulting in an overall larger diameter prosthesis as shown in FIG. 15C. As in the embodiments set forth above, the hardness of structural support pylon increases upon deployment.

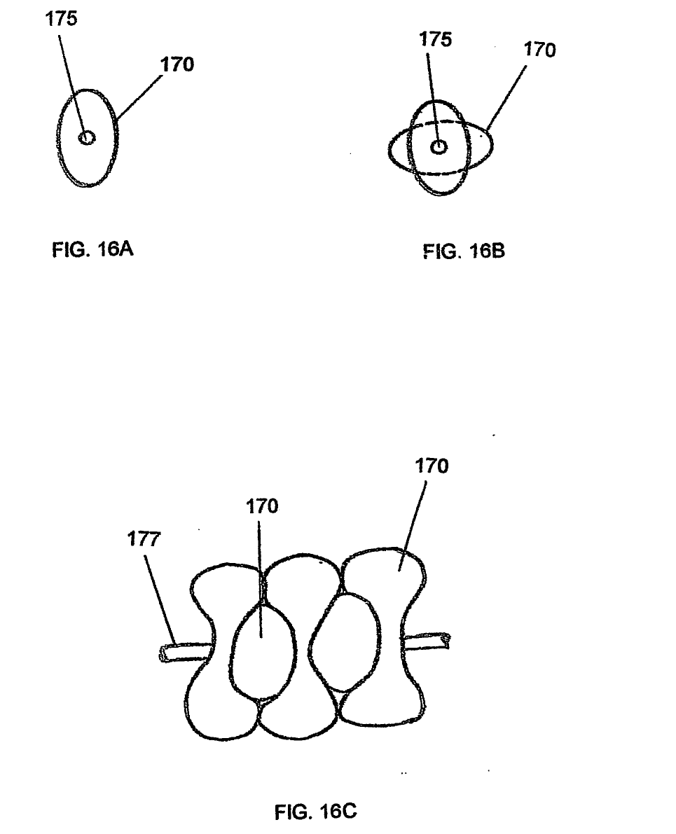

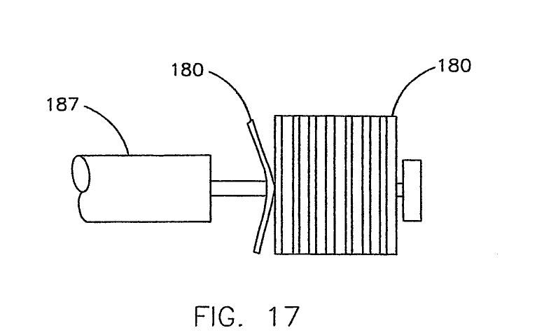

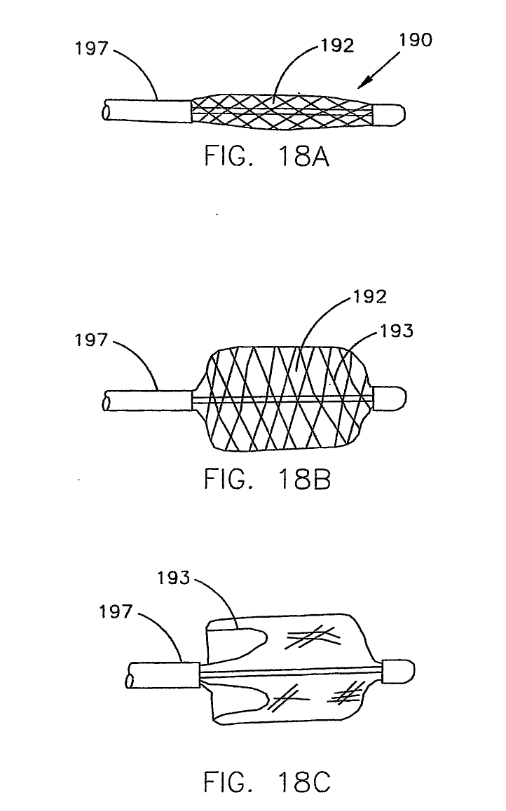

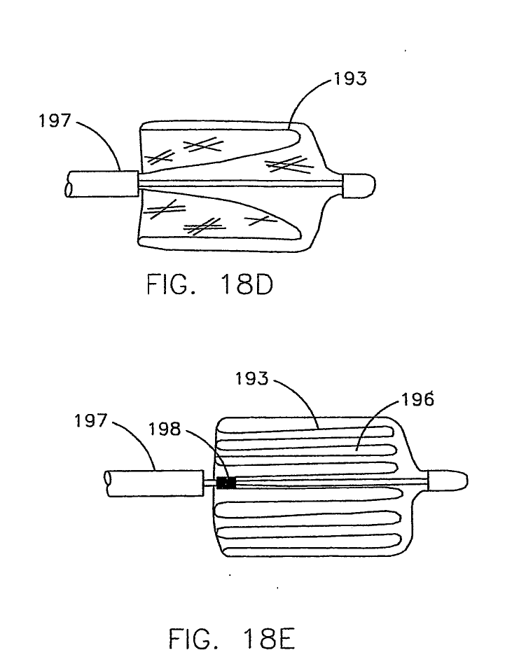

FIGS. 16A-16C illustrates the foregoing principles with respect to yet another alternative embodiment of structural support pylon 170. FIG. 16A illustrates a generally ovular shape and generally central aperture 175 in an end view prior to deployment. When mounted offset to one another at between 45-degree and 90-degree angles, the deployed configuration of a plurality of structural support pylons 170 is generally illustrated in a side view in FIG. 16C. As set forth above in relation to FIGS. 14A-15C, a greater expansion ratio can be achieved overall as a result of the offset of the individual support pylons. Accordingly, expansion ratios in the range of between one and one tenth (1.1) fold and eight (8) fold can be achieved.

Suitable materials for structural support pylons include but are not limited to biocompatible metals, alloys, including nickel titanium, polymers, ceramics, and composites thereof.

FIG. 17 illustrates a side view of another alternative embodiment according to the invention. The embodiment of FIG. 17 comprises a plurality of structural support discs 180, which are generally placed adjacent to one another upon deployment The delivery configuration of such an embodiment comprises independent discs mounted upon within delivery cannula 187, each disc folded conically or in another suitable configuration within delivery cannula 187. Following the creation of a delivery path 10 within a vertebral body according to any of the foregoing examples, delivery cannula 187 is placed at a desired location, and a pushing mandrel (not pictured) ejects structural support discs 180 from the end of delivery cannula 187, one by one. Structural support discs revert from a folded, delivery configuration, to a deployed configuration, thereby providing support in their multitude at a desired location within a diseased and/or injured vertebral body. Typically, the height of the foregoing embodiment can be increased by between one and one tenth (1.1) and ten (10) fold over its delivery configuration height.

Suitable materials for structural support discs include but are not limited to biocompatible metals, alloys, including nickel titanium, polymers, ceramics, and composites thereof

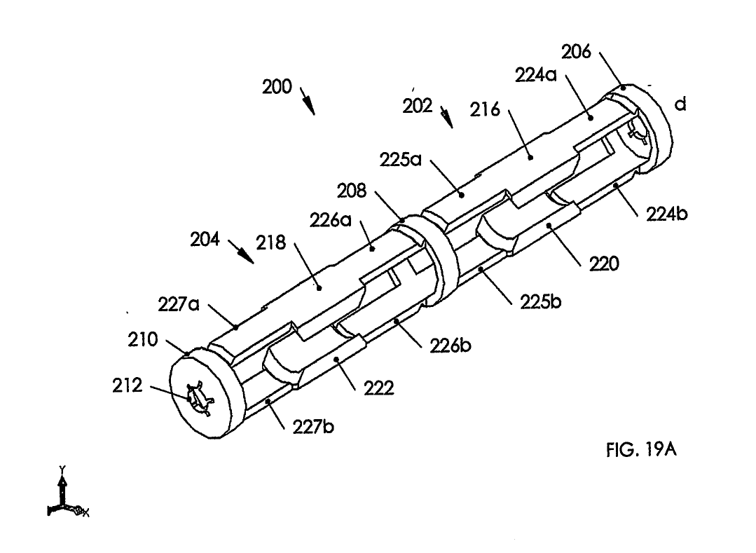

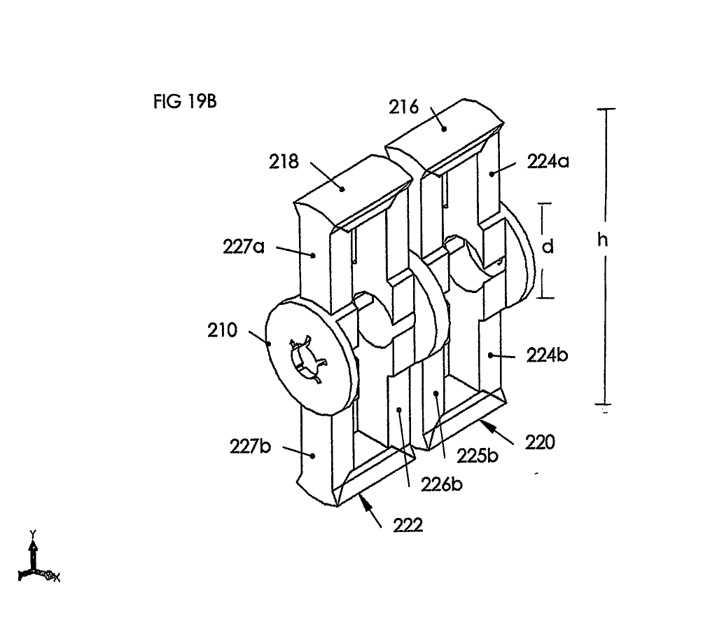

FIGS. 18A-18E illustrate yet another alternative embodiment of a structural support pylon 190. Structural support pylon 190, shown in its delivery configuration in FIG. 18A, comprises an “inverted” and layered braid structure. After a path of either generally semi-circular or other suitable configuration has been created within a vertebral body according to any of the foregoing descriptions, a first layer 192 of structural support pylon 190 is pushed from the distal end of delivery cannula 197. Fiber 193 of structural support pylon may be manufactured from any suitable material, and may comprise shape memory characteristics. FIGS. 18A and 18B depict first layer 192. Next, in order to continue deployment of structural support pylon 190, second layer 194 is formed within structured support pylon 190, by pushing additional fiber 193 through the distal end of cannula 197, as represented by the partial cross section of FIGS. 18C-18D (in which first layer 192 has been removed from view). The process may be repeated to form a third layer 196, as illustrated in FIG. 18E. Then, lock 198 is engaged in order to secure fiber 193 in the deployed configuration. Structural support pylon 190 thereby provides continuing support within a diseased and/or injured vertebral body. Alternatively, a non-inverted braid design may be used.

FIG. 19A depicts an alternative embodiment according to the invention in its elongated delivery configuration. Scaffold 200, which alternatively comprises a sole unit, in this instance comprises first unit 202 and second unit 204. Scaffold 200 may be delivered in a manner similar to that described with respect to FIGS. 11A-11C, or in a manner similar to that described in relation to FIGS. 13A-13B. In the alternative, scaffold 200 may be delivered percutaneously from a curving or a non-curving posterior approach, either singly or followed by delivery of a second pylon (not pictured) in, for example, a contralateral fashion. Scaffold 200 may be delivered, for example, via a cannula.

In its delivery configuration, first unit 202 and second unit 204 lie in an end to end configuration. First endplate 206, second endplate 208 and third endplate 210 are in communication with first unit 202 and second unit 204. Interior 212 of first actuator wheel 206, and likewise second and third actuator wheels, may be smooth, threaded, notched, or otherwise configured to engage an actuator arm (not pictured), which is correspondingly smooth, threaded, notched or otherwise configured. When engaged, an actuator arm is operated to convert scaffold 200 from its delivery configuration to its deployed configuration, pictured in FIG. 19B.

Scaffold 200 comprises superior surfaces 216 and 218 and inferior surfaces 220 and 222. Scaffold 200 further comprises sides 224, 225, 226 and 227, which are made up of portions 224a, 224b, 225a, 225b, 226a, 226b, 227a, and 227b. When scaffold 200 is in its delivery configuration, a portion 224a generally lies end to end with superior surface 216, while portion 224b of side 224 generally lies end to end with inferior surface 220. Similarly, a portion 225a of side 225 generally lies end to end with superior surface 216, and portion 225b generally lies end to end with inferior surface 220. In corresponding second unit 204, a portion 226a of side 226 lies generally end to end with superior surface 218, and portion 226b lies generally end to end with inferior surface 222.

Following deployment via an acting arm (not pictured), the configuration of scaffold is transformed into the deployed configuration depicted in FIG. 19B. In its deployed configuration, sides 224, 225, 226 and 227 are roughly at right angles to superior surfaces 216, 218 and inferior surfaces 220 and 222. Scaffold 200, previously of a height d, roughly equivalent to the height only of actuator wheels 206, 208 and 210, now comprises height h. Height his typically between one and one tenth (1.1) times and ten (10) times height d.

Further, when deployed within a vertebral body (not pictured), superior surfaces 216 and 218 exert an upward force against the bottom surface of a superior vertebral body endplate, and inferior surfaces 220 and 222 brace against the top surface of an inferior vertebral body endplate, thereby reinforcing and stabilizing the vertebral body and restoring the vertebral body to a normal height.

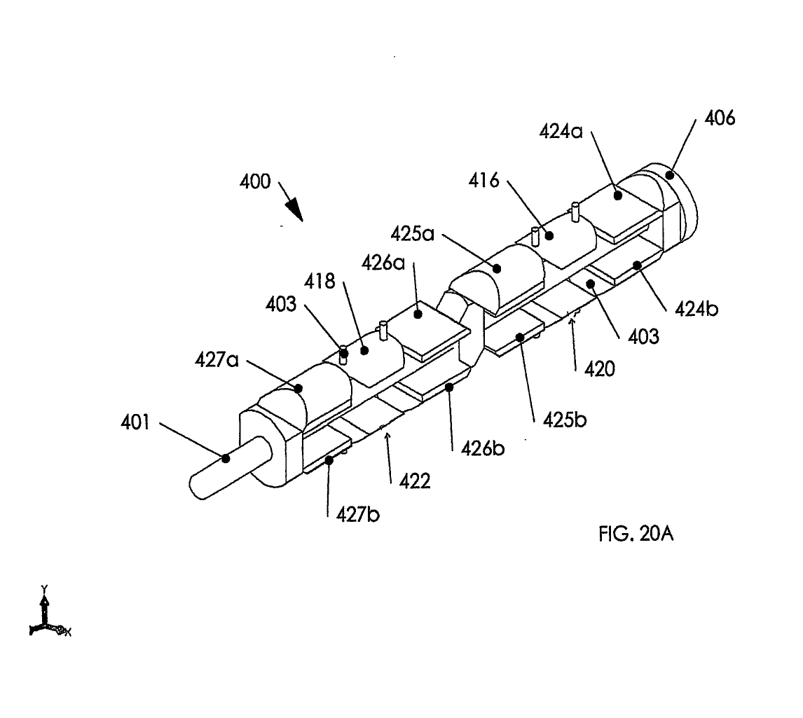

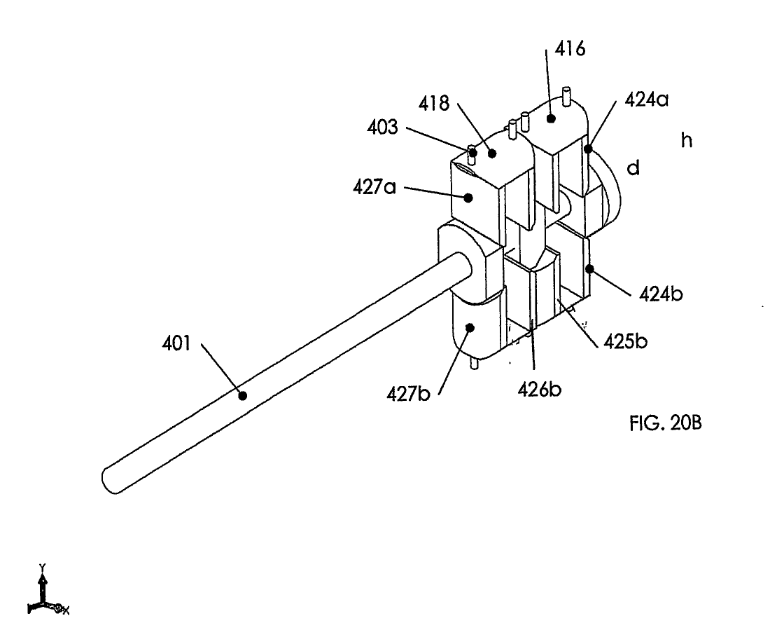

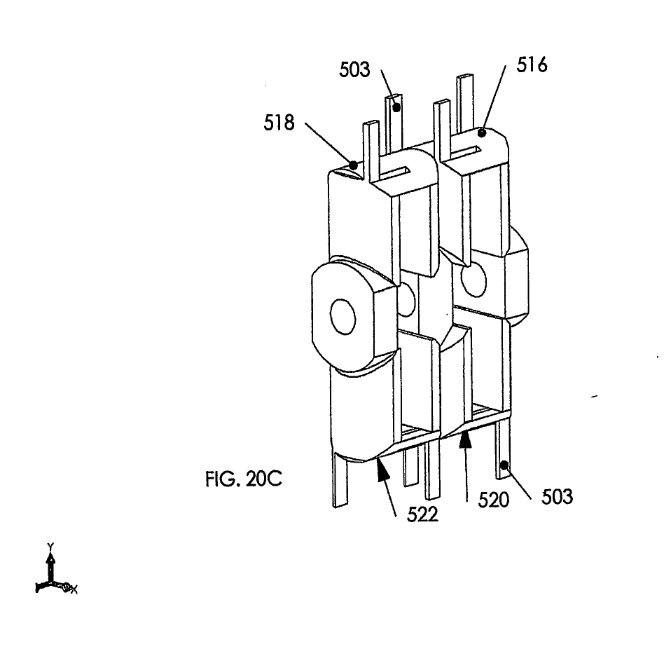

FIGS. 20A and 20B illustrate another variation on the foregoing embodiments. FIG. 20A depicts scaffold 400 mounted upon delivery mandrel 401. Following deployment, scaffold 400 comprises the configuration depicted in FIG. 20B. In its deployed configuration, side portions 424a, 424b, 425a, 425b, 426a, 426b, 427a, and 427b are roughly at right angles to superior surfaces 416, 418 and inferior surfaces 420 and 422.

Scaffold 400, previously of a height d, roughly equivalent to the height only of end plate 406, now comprises height h. Height h is typically between one and one tenth (1.1) times and ten (10) times height d. Further, similar to the embodiments set forth above, when scaffold 400 is deployed within a vertebral body (not pictured), superior surfaces 416 and 418 exert an upward force against the bottom surface of a superior vertebral body endplate, and inferior surfaces 420 and 422 brace against the top surface of an inferior vertebral body endplate, thereby reinforcing and stabilizing the vertebral body and restoring the vertebral body to a normal height. Further, pins 403, in their delivery configuration in FIG. 20A, protrude to their deployed configuration in FIG. 20B. In their deployed configuration, pins 403 protrude to engage the interior or the vertebral body and endplates, and serve to anchor scaffold 400 within the vertebral body.

FIG. 20C illustrates a similar embodiment according to the invention in its deployed configuration. Anchoring members 503 protrude from superior surfaces 516 and 518, and from inferior surfaces 520 and 522. Alternatively to pins or anchors, superior and inferior surfaces of the device may comprise surface roughness, barbs, adhesives, or other suitable means for securing the device within the vertebral body. Any of the foregoing embodiments according to the invention, can typically withstand compressive stresses in excess of between 700 and 1,000 Newtons, and cyclic loads in excess of 500-1200 Newtons for 10 million cycles. The embodiments set forth in FIGS. 19A-20C may be manufactured from stainless steel, titanium, nickel titanium or other suitable materials.

While particular forms of the invention have been illustrated and described above, the foregoing descriptions are intended as examples, and to one skilled in the art it will be apparent that various modifications can be made without departing from the spirit and scope of the invention For example, while the foregoing description sets forth examples of treatment of a vertebral body because of the high frequency of occurrence and degree of seriousness of injury to a vertebral body, it is apparent that the inventive concepts herein can be applied in other disease and/or sites of injury without departing from the spirit and scope of the invention.

1. An endoprosthesis for use in the treatment of diseased or fractured bone, said endoprosthesis comprising an elastomeric core, a delivery configuration and a deployed configuration, wherein said delivery configuration comprises a first width and a first height, and said deployed configuration comprises a second, reduced width and a second, increased height a reduced diameter configuration and an expanded diameter configuration.

2. The endoprosthesis according to claim 1, said endoprosthesis comprising sufficient outward radial strength to resist multi-directional loads, said multi-directional loads comprising compressive loads of between 50N and 6000N.

3. The endoprosthesis according to claim 1, said endoprosthesis further comprising one or more therapeutic substances.

4. The endoprosthesis according to claim 3, wherein said one or more therapeutic substances comprises bone morphogenic protein.

5. The endoprosthesis according to claim 3, wherein said one or more therapeutic substances is disposed about said endoprosthesis using a solvent in a supercritical state.

6. The endoprosthesis according to claim 1, wherein said delivery configuration comprises a generally cylindrical configuration and said deployed configuration comprises a generally ellipsoidal or a generally spherical configuration.

7. The endoprosthesis according to claim 1, wherein said elastomeric core comprises a first hardness when said endoprosthesis is in said delivery configuration, and a second hardness when said endoprosthesis is in said deployed configuration, wherein said second hardness is greater than said first hardness.

8. The endoprosthesis according to claim 7, wherein said first hardness is in the range of less than 10 Shore A durometer and said second hardness is in the range of 20 to 70 Shore A durometer.

9. The endoprosthesis according to claim 1, wherein said elastomeric core comprises an aperture therethrough.

10. The endoprosthesis according to claim 9, wherein said aperture is disposed centrally within said elastomeric core.

11. The endoprosthesis according to claim 9, wherein said aperture is disposed eccentrically within said elastomeric core.

12. The endoprosthesis according to claim 6, wherein said generally cylindrical configuration comprises one or more flat sides.

13. The endoprosthesis according to claim 6, wherein said generally cylindrical configuration comprises a transverse axis, and a generally ovular cross section along said transverse axis.

14. The endoprosthesis according to claim 9, wherein said aperture comprises a threaded interior for engagement with a threaded member.

15. The endoprosthesis according to claim 9, wherein said aperture comprises a notched interior for engagement with a ratcheting member.

16. The endoprosthesis according to claim 9, wherein said aperture comprises a smooth surface for engagement with a smooth mandrel.

17. The endoprosthesis according to claim 1, wherein said expanded diameter configuration is between 1.1 and 10 times the greater than said reduced diameter configuration.

18. An endoprosthesis for use in repairing a diseased or fractured bone, said endoprosthesis comprising endoprosthesis members, a substantially hollow interior and endplates, and comprising a delivery configuration and a deployed configuration.

19. The endoprosthesis according to claim 18, said endoprosthesis comprising sufficient outward radial strength to resist multi-directional loads, said multi-directional loads comprising compressive loads of between 50N and 6000N.

20. The endoprosthesis according to claim 18, said endoprosthesis further comprising one or more therapeutic substances.

21. The endoprosthesis according to claim 20, wherein said one or more therapeutic substances comprises bone morphogenic protein.

22. The endoprosthesis according to claim 20, wherein said one or more therapeutic substances is disposed about said endoprosthesis using a solvent in a supercritical state.

23. The endoprosthesis according to claim 18, wherein said endplates comprise a threaded interior for engagement with a threaded element.

24. The endoprosthesis according to claim 18, wherein said aperture comprises a notched interior for engagement with a ratcheting member.

25. The endoprosthesis according to claim 18, wherein said aperture comprises a smooth surface for engagement with a smooth mandrel.

26. The endoprosthesis according to claim 18, wherein said endoprosthesis members comprise preferential bending regions.

27. The endoprosthesis according to claim 18, wherein said delivery configuration is generally cylindrical and said deployed configuration is generally ellipsoidal or generally spherical.

28. The endoprosthesis according to claim 18, wherein said endoprosthesis comprises a central region and one or more end regions, and said deployed configuration is generally ellipsoidal or generally spherical about said central region, and generally cylindrical about said one or more end regions.

29. The endoprosthesis according to claim 18, wherein said deployed configuration is between 1.1 and 10 times greater than said delivery configuration.

30. An endoprosthesis for use in repairing a diseased or fractured bone, said endoprosthesis comprising a sleeve and one or more inverted end members and an interior, wherein said one or more inverted end members engage said sleeve within said interior, said endoprosthesis comprising a reduced profile configuration and an expanded profile configuration.

31. The endoprosthesis according to claim 30 wherein said inverted end members comprise a threaded interior for engagement with a threaded member.

32. An endoprosthesis for use in repairing diseased or fractured bone comprising a reduced profile delivery configuration and an expanded profile deployment configuration, said endoprosthesis comprising a superior end plate, an inferior endplate, and an expandable member disposed therebetween.

33. The endoprosthesis according to claim 32 wherein said expandable member comprises a threaded element.

34. The endoprosthesis according to claim 33 wherein said expandable member comprises a ratcheting element.

35. The endoprosthesis according to claim 33 wherein said expandable member is self-expanding.

36. An assembly for use in repairing diseased or injured bone, said assembly comprising one or more structural support pylons, wherein one or more of said structural support pylons comprises a delivery configuration and a deployed configuration, wherein said delivery configuration comprises a first width and a first height, and said deployed configuration comprises a second, reduced width and a second, increased height.

37. The assembly according to claim 36, wherein said assembly further comprises a structural reinforcement material.

38. The assembly according to claim 36, wherein said assembly further comprises a threaded element, wherein said plurality of structural support pylons are engaged with said threaded element.

39. The assembly according to claim 36, wherein said assembly further comprises a ratcheting element, and wherein said plurality of structural support pylons are engaged with said ratcheting element.

40. The assembly according to claim 36, wherein said assembly further comprises a smooth mandrel, and wherein said plurality of structural support pylons are engaged with said smooth mandrel.

41. The assembly according to claim 36, wherein said one or more of said plurality of structural support pylons are disposed at between 45 degree and 90 degree angles with respect to an adjacent structural support pylon.

42. An assembly for use in repairing diseased or fractured bone, said assembly comprising a plurality of structural support discs, wherein said assembly comprises a reduced profile delivery configuration and an expanded profile deployment configuration.

43. An endoprosthesis for use in repairing diseased or fractured bone, said endoprosthesis comprising a reduced profile delivery configuration and an expanded profile deployment configuration, wherein said endoprosthesis comprises an inverse layered braid configuration.

44. The endoprosthesis according to claim 43, wherein said endoprosthesis comprises means for locking said braid configuration.

45. An endoprosthesis for use in the treatment of diseased or fractured bone, said endoprosthesis comprising a delivery configuration and a deployed configuration, wherein said delivery configuration is generally cylindrical and said deployed configuration is generally ellipsoidal.

46. The endoprosthesis according to claim 45, wherein said endoprosthesis is deployed by mechanical means.

47. The endoprosthesis according to claim 46, wherein said mechanical means comprises a ratchet column.

48. The endoprosthesis according to claim 46, wherein said endoprosthesis comprises endplates, and wherein said mechanical means comprises a threaded element connecting said endplates.

49. The endoprosthesis according to claim 46, wherein said threaded member is disposed in the interior of said endoprosthesis.

50. An endoprosthesis for use in the treatment of diseased or fractured bone, said endoprosthesis comprising a delivery configuration and a deployed configuration, wherein said delivery configuration comprises a first width and a first height, and said deployed configuration comprises a second, reduced width and a second, increased height.

51. The endoprosthesis according to claim 50, wherein said second, increased height is between one and one tenth and ten times said first height.

52. The endoprosthesis according to claim 50, wherein said endoprosthesis comprises a plurality of folded discs when in said delivery configuration and a plurality of unfolded discs when in said deployed configuration.

53. The endoprosthesis according to claim 50, wherein said endoprosthesis comprises a superior surface, an inferior surface, and two or more side portions, wherein when in its delivery configuration, one or more of said side portions lie substantially in the same plane as the superior surface, and one or more of said side portions lie substantially in the same plane as the inferior surface, and when in its deployed configuration, one or more said side portions lie in a plane substantially perpendicular to said superior surface and one or more said side portions lie in a plane substantially perpendicular to said inferior surface.

54. The endoprosthesis according to claim 53, wherein one or more of said superior surface and inferior surface comprises means for engaging the interior of a vertebral body.

55. The endoprosthesis according to claim 54, wherein said means comprises a roughened surface.

56. The endoprosthesis according to claim 54, wherein said means comprises one or more protrusions.

57. The endoprosthesis according to claim 54, wherein said means comprises one or more adhesives.

58. The endoprosthesis according to claim 54, wherein said means comprises chemical means.

59. The endoprosthesis according to claim 54, wherein said means comprises one or more barbs.

60. An assembly for use in repairing diseased or fractured bone, said assembly comprising one or more structural support pylons and an actuating arm for the deployment of said one or more structural support pylons.

61. The assembly according to claim 60, wherein said assembly further comprises a structural reinforcement material.

62. The assembly according to claim 60, wherein said actuating arm is smooth.

63. The assembly according to claim 60, wherein said actuating arm is threaded.

64. The assembly according to claim 60, wherein said actuating arm is ratcheted.

65. The assembly according to claim 60, said assembly further comprising one or more washers.

66. A minimally invasive method for repairing diseased or fractured bone, said method comprising:
percutaneously introducing an endoprosthesis within the interior of said injured bone, said endoprosthesis comprising a delivery configuration and a deployed configuration, wherein said delivery configuration comprises a first width and a first height, and said deployed configuration comprises a second, reduced width and a second, increased height.

67. The method according to claim 66, wherein a generally semi-circular path is created within said bone prior to introducing said endoprosthesis.

68. The method according to claim 67, wherein said generally semi-circular path is oriented vertically within said bone.

69. The method according to claim 67, wherein said generally semi-circular path is oriented laterally within said bone.

70. The method according to claim 66, wherein said endoprosthesis is generally cylindrical, comprises an interior, and said step of deploying said generally cylindrical endoprosthesis comprises pulling a tapered plug through said interior.

71. The method according to claim 66, said method further comprising the step of administering a therapeutic within said diseased or fractured bone.

72. The method according to claim 66, said method further comprising the step of introducing a structural support material within said diseased or injured bone.

73. The method according to claim 66, wherein said endoprosthesis comprises first and second ends, wherein said first and second ends are at a first distance apart when said endoprosthesis is in its delivery configuration, and at a second, lesser distance apart when said endoprosthesis is in its deployed configuration, and wherein said step of deploying said endoprosthesis comprises decreasing said first distance.

74. The method according to claim 66, wherein said endoprosthesis comprises endoprosthesis members and voids therebetween, and wherein said endoprosthesis members comprise one or more preferential bending regions.

75. The method according to claim 74, wherein said endoprosthesis comprises a threaded member engaging said first and second ends, and said step of decreasing said first distance comprises advancing one of said first and second ends over said threaded member.

76. The method according to claim 75, wherein said endoprosthesis comprises an interior, and said threaded member is disposed within said interior.

77. The method according to claim 74, wherein said endoprosthesis comprises a ratchet column, and said step of decreasing said first and second distance comprises actuating said ratchet column.

78. The method according to claim 74, wherein said endoprosthesis comprises a smooth mandrel engaging said first and second ends, and said step of decreasing said first distance comprises advancing said of said first and second ends over said smooth mandrel.
